(19) United States
(12) Patent Application Publication
(10) Pub. No.: US 2026/0131346 A1
(43) Pub. Date: May 14, 2026

(54) METHOD AND DEVICE FOR COATING A MEDICAL INVASIVE COMPONENT

(71) Applicant: B. Braun Melsungen AG, Melsungen (DE)

(72) Inventor: Eberhard Engelien, Wohltorf (DE)

(21) Appl. No.: 19/478,994

(22) PCT Filed: Apr. 26, 2024
(86) PCT No.: PCT/EP2024/061599

§ 371 Date: Oct. 27, 2025

(30) Foreign Application Priority Data
Apr. 28, 2023 (DE) 10 2023 111 000.4

(51) Int. Cl. B05C 5/02; A61L 29/08; B05C 1/02; B05C 9/14; B05C 11/10; B05C 13/02

(52) U.S. Cl. CPC B05C 5/0216; A61L 29/085; B05C 1/022; B05C 5/0241; B05C 5/027; B05C 9/14; B05C 11/1007; B05C 11/1036; B05C 11/1044; B05C 13/025; A61L 2420/02

(57) ABSTRACT

An apparatus for coating a medical component has a rotation device configured to clamp and rotate the medical component with a defined rotational speed. An application device is rotationally immovable and configured to apply a viscous coating solution onto a lateral face of the rotating medical component with a defined application rate. A linear movement device is configured to move linearly between the application device and the rotation device with a defined advancing speed. A control device is configured to control the rotational speed, the application rate, and the advancing speed in a manner coordinated to one another depending on the viscosity of the viscous coating solution such that the viscous coating solution can be applied over the full surface of the lateral face in the form of a helix which overlaps along the longitudinal axis.

### CROSS-REFERENCE TO RELATED APPLICATIONS

This application is the United States national phase entry of International Application No. PCT/EP 2024/061599, filed on Apr. 26, 2024, and claims priority to German Application No. 10 2023 111 000.4, filed on Apr. 28, 2023.

### FIELD

The invention relates to a method and an apparatus for coating a medical invasive component.

### BACKGROUND

Medical invasive components are often provided with a coating in order to improve their functional properties.

For example, catheter tubes are usually provided with a hydrophilic coating, which forms a sliding film upon contact with fluid, which sliding film is intended to improve the sliding properties of the catheter tube in a guide tube or in the interior of the body.

Different methods for the hydrophilic coating of catheter tubes are known from the prior art.

In a known method, a viscous coating solution is sprayed onto the lateral face of the catheter tube. The viscous coating solution comprises a volatile solution component, after the evaporation of which the actual coating material remains on the lateral face in the form of the hydrophilic coating. Regions of the lateral face that are not to be coated must be covered over before the spraying. In addition, in such spraying methods, more viscous coating solution has to be used than ultimately lands on the lateral face.

In another known method, the catheter tube is immersed in the viscous coating solution, which in turn requires that regions not to be coated are covered over. Even in such immersion methods, a significantly larger amount of the viscous coating solution has to be provided than ultimately reaches the lateral face.

In another known method, the viscous coating solution is painted onto the lateral face using a tool, for example a sponge or brush. The amount of coating solution that can be painted on is limited by the contact force of the tool. The layer thickness of the coating is not adjustable or is difficult to adjust.

### SUMMARY

The object of the invention is to provide a method and an apparatus allowing improved coating of medical invasive components that have a rotationally symmetrical lateral face extending in a straight manner. In particular, the aim is to ensure that selected regions are coated over the greatest possible surface area with an easily adjustable layer thickness, with minimized consumption of viscous coating solution.

The apparatus according to the invention comprises a rotation device, an application device, a linear movement device and a control device. The rotation device is configured to clamp the invasive component and rotate the latter in a driven manner about its longitudinal axis. The rotation device is configured to rotate the invasive component at a defined rotational speed. The rotational speed is a measure of the number of revolutions of the rotation device and thus also of the invasive component per unit of time. The application device is rotationally immovable relative to the longitudinal axis and is configured to apply a viscous coating solution to the lateral face of the rotating invasive component. The application device is configured to apply the viscous coating solution at a defined application rate. The application rate is a measure of a volume or weight of the viscous coating solution applied per unit of time. In one embodiment, the application device is arranged, with respect to the gravitational direction, below the longitudinal axis (“standing drop of the viscous coating solution”). In a further embodiment, the application device is instead arranged above the longitudinal axis (“hanging drop of the viscous coating solution”). The linear movement device is configured for the driven linear movement of the application device and/or of the rotation device along the longitudinal axis of the rotating invasive component. By means of the linear movement device, the application device and the rotation device are driven relative to each other in a linear movement. In one embodiment, the linear movement device is configured for the linear movement of the application device, with the rotation device being linearly immovable. In a further embodiment, the linear movement device is configured for the linear movement of the rotation device, with the application device being linearly immovable. In a further embodiment, the linear movement device is configured for the linear movement of both the application device and the rotation device. The linear movement device is configured for linear movement at a defined speed of advance. The speed of advance is a measure of a distance traveled by the application device and/or the rotation device per unit of time along the longitudinal axis. The control device is configured to control the rotational speed of the rotation device, the application rate of the application device and the speed of advance of the linear movement device. The control is automatic. For this purpose, the control device can be connected to the rotation device, application device and linear movement device wirelessly or by wire. For applying the coating, the invasive component is first clamped into the rotation device. For clamping the invasive component, the rotation device is configured in different ways in different embodiments. For example, the rotation device can have a clamping unit with movable clamping jaws or the like. Thereafter, the clamped invasive component is rotated about its longitudinal axis. For this purpose, the rotation device preferably has a drive unit, in particular an electric drive. However, the drive unit is not part of the apparatus in all embodiments.

Furthermore, the application device and/or the rotation device are moved by means of the linear movement device under simultaneous application of the coating solution along the longitudinal axis of the rotating invasive component. For applying the viscous coating solution at the defined application rate, the application device in one embodiment has at least an application unit and a pump unit. The optional application unit can be designed, for example, in the form of a hollow needle, provided with an outlet opening at one end, a tube, a tapering hose or the like and serves for applying the viscous coating solution onto the lateral face. The application device, in particular the application unit, is preferably at such a radial distance from the longitudinal axis that the rotating lateral face as it were draws the viscous coating solution off from the application device/application unit. The distance is preferably between 0.1 mm and 1.5 mm, particularly preferably between 0.2 mm and 1.0 mm. Should the lateral face to be coated have an outer diameter that varies along the longitudinal axis, the distance is preferably kept constant, for example by means of a radial linear movement device which is configured for relative radial displacement between the application device and the rotation device. The optional pump unit serves for conveying the viscous coating solution through the application unit, wherein the application rate can be influenced by a change in a pumping output of the pump unit. The pumping output and thus the application rate is preferably controlled such that the application device, in particular the application unit, produces a (standing or hanging) drop which is drawn off upon contact with the rotating lateral face, as a result of which the viscous coating solution winds around the invasive component. In a further embodiment, the pump unit is arranged away from the application device and consequently in a stationary position relative thereto. For example, the pump unit can be arranged in the region of a solution container, which stores the viscous coating solution to be applied. The movement is effected by means of the linear movement device. The viscous coating solution is applied or wound in a spiral shape, i.e. helically, onto the lateral face moving in rotation relative to the application device. The linear movement of the application device can take place over an entire length of the lateral face or only over a part, i.e. selected regions, thereof. The viscous coating solution applied in this way to the lateral face forms the actual coating after drying, evaporating and/or crosslinking. Whether drying, evaporating and/or crosslinking of the viscous coating solution takes place in order to form the coating depends on the properties of the coating solution/coating used and is not essential to the core of the invention. The homogeneity, full-surface coverage and/or layer thickness of the coating can be easily adjusted by coordinated control of the rotational speed, application rate and speed of advance. In addition, it is conceivable and possible for multiple advances to take place in opposite directions of advance, such that the coating is as it were formed by several layers of the viscous coating solution. According to the invention, the control device is configured to control the rotational speed, the application rate and the speed of advance as a function of the viscosity of the viscous coating solution. The viscosity of the viscous coating solution forms a control parameter of the automatic control. In one embodiment of the invention, the apparatus has an input device which is configured for manual entry of an input variable representing the viscosity. In a further embodiment of the invention, the apparatus has a storage device in which data are stored, which data are representative of different viscosities of different coating solutions. The data can be retrieved from the storage device depending on the viscous coating solution actually used and can serve as a basis for the control. It is also conceivable and possible that the viscosity is determined by means of a measuring device during operation of the apparatus. If the lateral face has an outer diameter that varies along the longitudinal axis, the control device is preferably configured to control the rotational speed, the application rate and the speed of advance as a function of the viscosity and the changing outer diameter. In this case, the optional storage device can store data which are representative of the outer diameter that varies along the longitudinal axis. Furthermore, according to the invention, the control device is configured for coordinated control of the rotational speed, the application rate and the speed of advance, in such a way that the viscous coating solution can be applied to the entirety of the lateral face in the form of a helix which overlaps along the longitudinal axis. The rotational speed, the application rate and the speed of advance are thus coordinated with one another in such a way that the lateral face is continuously covered by the viscous coating solution in the circumferential direction and axial direction. In one embodiment, the coordinated control is based on a predetermined rotational speed, wherein the application rate and/or the speed of advance are accordingly adapted thereto, for example on the basis of corresponding determination equations, based on characteristic diagrams or the like. In a further embodiment, the coordinated control is based on a predetermined application rate, wherein the rotational speed and the speed of advance are determined accordingly. In a further embodiment, the coordinated control is based on a predetermined speed of advance, wherein the rotational speed and/or the application rate are accordingly adapted. It goes without saying that combinations of the aforementioned approaches are also conceivable and possible. Within the scope of this description, value ranges defined by formulations such as “between value X and value Y” expressly also include their limit values X and Y, which are stated here only by way of example. Preferably, the lateral face to be coated has an outer diameter of between 0.5 mm and 1.5 mm, preferably of between 0.7 mm and 1.0 mm, particularly preferably of between 0.8 mm and 0.9 mm. The outer diameter is preferably constant over the longitudinal axis.

The solution according to the invention is suitable for coating medical invasive components having a longitudinal axis and a lateral face which is rotationally symmetrical about the longitudinal axis and extends in a straight manner. Particularly preferably, the solution according to the invention is suitable for hydrophilic coating of catheter tubes.

In a further embodiment of the invention, the viscous coating solution to be applied is formed from a plurality of separately stored solution components, wherein the application device is configured for simultaneous application of the plurality of solution components and has an application unit for each of the plurality of solution components. This embodiment of the invention allows simple and efficient application of multi-component coating solutions. The application units are preferably each in the form of a hollow needle or the like provided with an outlet opening at one end. The different solution components do not necessarily have to be applied simultaneously. Rather, it is conceivable and possible that one of the solution components is first of all applied along a direction of advance, and thereafter another solution component is applied along an opposite direction of advance. Alternatively, the application is carried out successively in the same direction of advance, as a result of which it can be ensured in a simple way that coated regions of the lateral face receive the same evaporation and/or drying times before a further solution component is applied. It is possible in principle that multi-component coating solutions can also be applied using just one application unit. However, in some cases this requires intermediate cleaning of the application unit. This can be dispensed with in this embodiment of the invention.

In a further embodiment of the invention, an evaporation device is present and is configured for evaporating and/or accelerating evaporation of a volatile solution component of the viscous coating solution applied to the lateral face. In particular, hydrophilic coating solutions often have a volatile solution component, which serves as solvent for an actual coating material. After the viscous coating solution has been applied, an evaporation of the volatile solution component is provided, so that the coating material remains on the lateral face and forms the hydrophilic coating. The evaporation device causes and/or supports this evaporation process. In different embodiments, the evaporation device is designed differently. In one embodiment, the evaporation device has a heating unit for heating the lateral face provided with the viscous coating solution. In a further embodiment, the evaporation device alternatively or additionally has a fan unit, which is configured to blow onto the lateral face provided with the viscous coating solution. By heating and/or blowing on the viscous coating solution located on the lateral face, the evaporation process can be initiated, maintained and/or accelerated depending on the specific properties of the viscous coating solution used and on the ambient conditions.

In a further embodiment of the invention, a crosslinking device is present and is configured for crosslinking and/or accelerating crosslinking of crosslinkable solution components of the viscous coating solution applied to the lateral face. In particular, hydrophilic coatings are often formed with crosslinking of viscous solution components. The solution components used are preferably crosslinked individually and successively and not together. The crosslinking device is configured for initiating, maintaining and/or accelerating the crosslinking process and is designed differently in different embodiments of the invention. For example, coating solutions are known that are crosslinkable by light, in particular UV light. In this case, the crosslinking device preferably has at least one light source, in particular a UV light source.

In a further embodiment of the invention, the rotation device is configured for the clamping and driven rotation of invasive components having a diameter of 0.5 mm to 10 mm, preferably of 0.7 mm to 0.9 mm, and a length of 40 mm to 500 mm, preferably of 80 mm to 250 mm, and having a rotational speed of 20 rpm to 900 rpm, preferably of 250 rpm to 800 rpm, preferably having a rotational speed of 300 rpm to 600 rpm, preferably of 600 rpm.

In a further embodiment of the invention, the application device is configured to apply viscous coating solutions having a viscosity of 25×10⁻³ kg/ms to 80×10⁻³ kg/ms, preferably of 35×10⁻³ kg/ms to 55×10⁻³ kg/ms, more preferably of 43×1⁻³ kg/ms to 48×10⁻³ kg/ms, with an application rate of 0.5 μl/s to 10 μl/s, preferably of 0.7 μl/s to 5 μl/s, more preferably of 1 μl/s to 3 μl/s, more preferably of 0.9 μl/s to 2.5 μl/s. The processable viscosities therefore range between 25 and 80 cP (centipoise).

In a further embodiment of the invention, the linear movement device is configured for the driven linear movement of the application device and/or the rotation device with a speed of advance of between 0.5 mm/s and 50 mm/s, preferably of between 4 mm/s and 40 mm/s, more preferably of between 5 mm/s and 10 mm/s, more preferably 10 mm/s.

The method according to the invention serves for coating a medical invasive component having a longitudinal axis and a lateral face which is rotationally symmetrical about the longitudinal axis and extends in a straight manner. In particular, the method serves for hydrophilic coating of a catheter tube. The method according to the invention comprises the steps of: clamping the invasive component into a rotation device; rotating the clamped invasive component by means of the rotation device, the rotation taking place at a defined rotational speed; applying a viscous coating solution to the lateral face of the rotating invasive component, wherein the viscous coating solution is applied by means of an application device, which is rotationally immovable relative to the longitudinal axis, and at a defined application rate; linearly moving the application device and/or the rotation device along the longitudinal axis of the rotating invasive component, wherein the application device is moved linearly by means of a linear movement device and at a defined speed of advance relative to the rotation device, and/or vice versa; controlling the rotational speed, the application rate and the speed of advance by means of a control device, wherein the rotational speed, the application rate and the speed of advance are controlled in a coordinated manner depending on the viscosity of the viscous coating solution, such that the viscous coating solution is applied over the full surface of the lateral face in the form of a helix which overlaps along the longitudinal axis. What has already been stated with respect to the apparatus according to the invention also applies, mutatis mutandis, to the method according to the invention. Therefore, to avoid repetition, reference is made expressly to the description of the apparatus according to the invention. Embodiments of the method according to the invention derive directly and unambiguously from the disclosure of the embodiments of the apparatus. Thus, embodiments of the method according to the invention can include, for example, application, evaporation and/or crosslinking.

In a further embodiment of the invention, the rotational speed is between 20 rpm and 900 rpm, preferably between 250 rpm and 800 rpm, preferably between 300 rpm and 600 rpm, preferably 600 rpm.

In a further embodiment of the invention, the viscosity is between 25×10⁻³ kg/ms and 80×10⁻³ kg/ms, preferably between 35×10⁻³ kg/ms and 55×10⁻³ kg/ms, more preferably between 43×10⁻³ kg/ms and 48×10⁻³ kg/ms.

In a further embodiment of the invention, the application rate is between 0.5 μl/s and 10 μl/s, preferably between 0.7 μl/s and 5 μl/s, more preferably between 1 μl/s and 3 μl/s, more preferably between 0.9 μl/s and 2.5 μl/s.

In a further embodiment of the invention, the speed of advance is between 0.5 mm/s and 50 mm/s, more preferably between 4 mm/s and 40 mm/s, preferably between 5 mm/s and 10 mm/s, more preferably 10 mm/s.

In a further embodiment of the invention, the rotational speed is between 300 rpm and 600 rpm, the viscosity is between 35×10⁻³ kg/ms and 55×10⁻³ kg/ms, the application rate is between 1 μl/s and 3 μl/s, and the speed of advance is between 5 mm/s and 10 mm/s. This is a particularly preferred embodiment. In a further embodiment of the invention, the rotational speed is 600 rpm, the viscosity is between 43×10⁻³ kg/ms and 48×10⁻³ kg/ms, the application rate is between 0.9 μl/s and 2.5 μl/s, and the speed of advance is 10 mm/s. The aforementioned value ranges of the process parameters have proven particularly advantageous in terms of solving the problem in question. This embodiment of the invention ensures in particular that, despite the comparatively highly viscous and/or pasty properties of the coating solution, no defects remain between adjacent windings of the helix/coating, and a constant and/or homogeneous layer thickness of the coating results along the longitudinal axis. Such coordinated control of the process parameters would not be necessary in the case of a comparatively low-viscosity coating liquid, since the latter is wetting and/or runs on the lateral face and can therefore be applied in the form of a liquid film-and not, for example, in the form of a helix.

In a further embodiment of the invention, coating with a primer, which can also be referred to as a base coat, is first carried out, and subsequently coating with a topcoat is carried out.

In a further embodiment of the invention, in relation to the gravitational direction, the viscous coating solution is discharged from downward in the form of a hanging drop on the application device, wherein the hanging drop is contacted by the rotating invasive component, as a result of which the viscous coating solution winds helically around the lateral face. The application rate is controlled in a coordinated manner for discharging the hanging drop.

In a further embodiment of the invention, in relation to the gravitational direction, the viscous coating solution is discharged upward in the form of a standing drop on the application device, wherein the standing drop is contacted by the rotating invasive component, as a result of which the viscous coating solution winds helically around the lateral face. The application rate is controlled in a coordinated manner for discharging the hanging drop.

### BRIEF DESCRIPTION OF THE DRAWINGS

Further advantages and features of the invention will become clear from the following description of preferred exemplary embodiments of the invention, which are illustrated with the aid of the drawings.

FIG. 1 shows a schematically simplified illustration of an embodiment of an apparatus according to the invention for coating a medical invasive component in the form of a catheter tube;

FIG. 2 shows, in enlarged detail, a section of the catheter tube;

FIG. 3 shows a schematically simplified illustration of a variant of an application device of the apparatus according to FIG. 1; and

FIG. 4 shows a flowchart for illustrating an embodiment of a method according to the invention.

### DETAILED DESCRIPTION

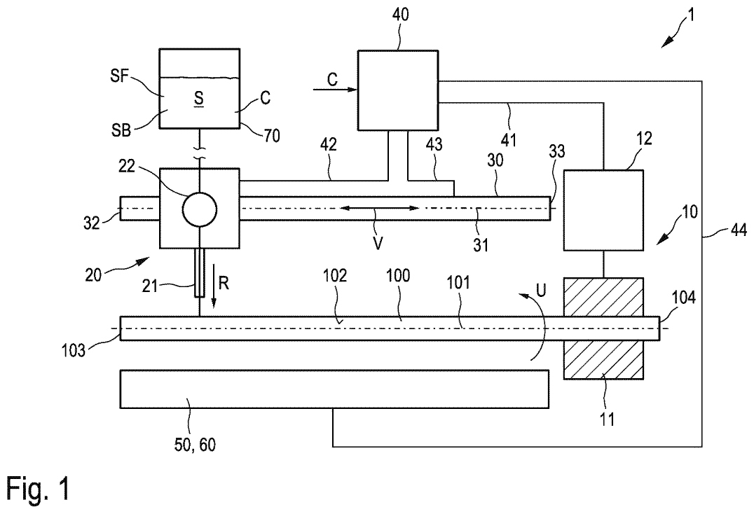

According to FIG. 1, an apparatus 1 is provided for coating a medical invasive component.

The invasive component in the present case is a catheter tube 100 having a longitudinal axis 101 and a lateral face 102. The lateral face 102 extends straight along the longitudinal axis 101 and is rotationally symmetrical. The catheter tube 100 to be coated extends between a first end 103 and a second end 104; the dimensions that can be seen from FIG. 1 are to be understood as purely exemplary and not to scale.

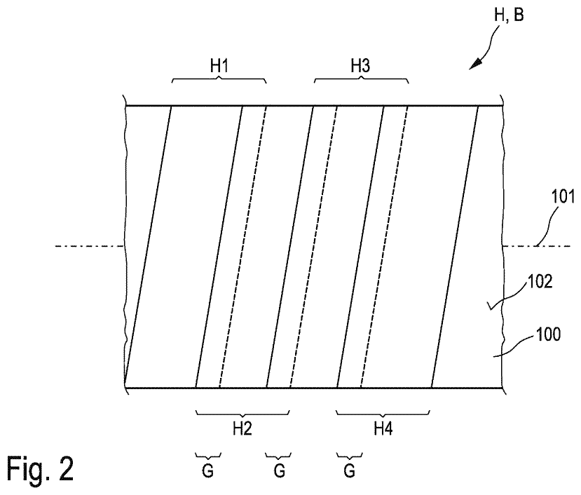

The coating to be applied by means of the apparatus 1 (see FIG. 2) is in the present case a hydrophilic coating B, which forms a sliding film upon contact with liquid. The sliding film supports mobility of the catheter tube 100 in the catheter system, such that the catheter tube 100 can be advanced as smoothly as possible to its destination in a guide tube and/or in the interior of the body. The coating B is formed, in a manner to be described in more detail, on the lateral face 102 from a viscous coating solution S (see FIG. 1).

The apparatus 1 comprises a rotation device 10, an application device 20, a linear movement device 30 and a control device 40.

In the embodiment shown, the apparatus 1 also has an optional evaporation device 50, which can alternatively also be configured as a crosslinking device 60. In addition, the apparatus in the present case has an optional solution container 70.

The rotation device 10 is configured for the clamping and driven rotation of the catheter tube 100 about its longitudinal axis 101. The rotation takes place at a defined rotational speed U, i.e. number of revolutions per unit of time.

The application device 20 is rotationally immovable relative to the longitudinal axis 101 and is configured to apply the viscous coating solution S to the lateral face 102 of the rotating catheter tube 100. The viscous coating solution S is applied at a defined application rate R, which represents a measure of the amount (volume and/or mass) of the coating solution S applied per unit of time.

The linear movement device 30 in the present case is configured for the driven linear movement of the application device 20 along the longitudinal axis 101. The linear movement is effected at a defined speed of advance V, i.e. distance along the longitudinal axis 101 per unit of time. In an embodiment not shown in the figures, the linear movement device is alternatively or additionally configured for the driven linear movement of the rotation device along the longitudinal axis.

The control device 40 is configured to control the rotational speed U of the rotation device 100, the application rate R of the application device 20 and the speed of advance V of the linear movement device 30. The rotational speed U and/or the speed of advance V can also be controlled as a function of an outer diameter of the catheter tube 100 and/or the lateral face 102, which outer diameter in particular varies over the longitudinal axis.

The viscous coating solution S is applied to the lateral face 102 during the advance movement of the application device 20 and upon the rotation of the rotation device 10 and thus of the catheter tube 100. Depending on how far the application device 20 is advanced along the longitudinal axis 101, the lateral face 102—with the exception of a portion clamped in the rotation device 10—can be provided substantially completely with the viscous coating solution S. However, it is also possible for only a longitudinal section of the lateral face 102 to be coated. In addition, it is conceivable and possible that the advance movement is repeated several times and/or carried out in opposite directions. This allows the viscous coating solution S to be applied in several layers.

In the embodiment shown, the rotational speed U, the application rate R and the speed of advance V are controlled as a function of the viscosity C of the viscous coating solution S to be applied. The viscosity C acts in this respect as a control parameter for the control device 40, although further or alternative control parameters can of course also be provided.

The viscosity C, more precisely a numerical value representing this physical parameter, can be manually entered, for example, on an input device not shown in FIG. 1. Alternatively or additionally, the viscosity C can be retrieved on the basis of data from a storage device or measured by means of a measuring device.

In the embodiment shown, the control device 40 is configured for coordinated control of the rotational speed U, the application rate R and the speed of advance V, specifically in such a way that the viscous coating solution S can be applied to the lateral face 102 in the form of a helix H overlapping along the longitudinal axis 101 (see FIG. 2).

The helix H can also be referred to as a coil or spiral and, in the schematic detailed representation according to FIG. 2, has four exemplary windings or turns H1, H2, H3, H4. Directly adjacent turns overlap along the longitudinal axis 101 by a dimension G, which can also be referred to as overlap. The overlap results in a full-surface coating.

In the embodiment shown, the viscous coating solution S, in relation to the gravitational direction, is discharged from the top downward in the form of a hanging drop (not shown in any detail in the figures) on the application device 20. The hanging drop is contacted by the rotating catheter tube 100 so that the viscous coating solution S winds helically around the lateral face 102. As a result, the coating solution S is not dripped or sprayed onto the lateral face 102, but rather “pulled away” from the application device.

The actual coating B, which in the present case is hydrophilic, is in the present case formed after drying and/or crosslinking of the viscous coating solution S applied to the lateral face 102.

In the embodiment shown in FIG. 1, the viscous coating solution S comprises a volatile solution component SF and a coating material SB dissolved in the latter. After evaporation of the volatile solution component SF, the coating material SB remains and forms the coating B on the lateral face 102.

In the embodiment shown, the rotation device comprises a clamping unit 11 and a drive unit 12.

The clamping unit 11 is configured to clamp the end of the catheter tube 100 and for this purpose, for example, has a movable chuck, a clamping device or locking device.

The drive unit 12 serves to rotate the clamping unit 11 and is preferably designed as an electric motor.

In addition, at least one support unit can be present and in particular can be assigned to the rotation device. The optional support unit provides radial support for the catheter tube to be coated and counteracts deflection of the catheter tube.

In the present case, for controlling the rotational speed U, the control device 40 is connected by means of a signal line 41 to the rotation device 10, in particular the drive unit 12. In the embodiment shown, the application device 20 has an application unit 21 and a pump unit 22.

The application unit 21 in the present case is in the form of a hollow needle and has an outlet opening (without reference sign) through which the viscous coating solution S exits from the application device 20 onto the lateral face 102. In the present case, said (hanging) drop is discharged at the outlet opening.

The pump unit 22 serves to pump the viscous coating solution S through the application unit 21 and the outlet opening thereof. In the embodiment shown, the pump unit 22 is assigned to the application device 20, although this is not the case in all embodiments. The pump unit 20 is connected to the solution container 70 via a fluid line (without reference sign). The viscous coating solution S to be applied is stored in the solution container 70. As already mentioned at the beginning, the solution container 70 is optional and is not present in all embodiments of the apparatus. Said fluid line is designed and arranged in such a way that a functional mobility of the application device 20 in relation to the stationary solution container 70 is guaranteed.

In the present case, the control device 40 for controlling the application rate R is connected to the application device 20 by means of a signal line 42. The application rate R in the present case is controlled, in particular adapted to the viscosity C and/or the further process parameters, in such a way that the aforementioned (hanging) drop forms and is drawn off continuously from the application device 20 onto the lateral face 102.

In the embodiment shown, the linear movement device 30 has a guide axis 31 which runs parallel to the longitudinal axis 101 and which extends between a first end 32 and a second end 33. The application unit 20 is guided and driven in a linear motion on the guide axis 31 between the first end 32 and the second end 33. The drive can be effected in any appropriate way. For example, the linear movement device 30 can interact with the application device 20 via a movement spindle, a belt drive, a rack or the like.

For controlling the speed of advance V, the control device 40 in the present case is connected to the linear movement device 30 via a signal line 43.

In the embodiment shown, the apparatus 1 also has the aforementioned, optional evaporation device 50. The evaporation device 50 supports the drying of the viscous coating solution S applied to the lateral face 102, by means of the process of evaporation of the volatile solution component SF being set in motion, maintained and/or accelerated. For this purpose, the evaporation device 50 can have a heating unit (not shown in any detail in the figures), in particular an infrared radiator, wherein alternatively or additionally a fan unit can also be present. In the embodiment shown, the evaporation device 50 extends substantially over an entire length of the possible path of advance of the application device 20 and is arranged on a side of the longitudinal axis 101 of the catheter tube 100 lying opposite the application device 20 and the linear movement device 30. The arrangement is stationary. In an embodiment not shown in the figures, the evaporation device can instead be comparatively shorter, arranged movably together with the application device and offset from the latter by a certain longitudinal distance. This results in what is by comparison a more compact design.

In the embodiment shown, the evaporation device 50 is integrated into the control of the apparatus 1 and for this purpose is connected to the control device 40 via a signal line 44.

In the embodiment shown, the rotation device 10 is configured for the clamping and rotating of invasive components that have a diameter of 0.5 mm to 10 mm, in particular of 0.7 mm to 0.9 mm. The length of the coatable invasive components is from 40 mm to 500 mm, in particular from 80 mm to 250 mm. The possible rotational speed U is between 20 rpm and 900 rpm.

In the embodiment shown, the application device 20 allows the processing of viscous coating solutions having a viscosity V of 25 cP to 80 cP, which corresponds to 25×10⁻³ kg/ms to 80×10⁻³ kg/ms. The possible application rates range from 0.5 μl/s to 10 μl/s.

Furthermore, speeds of advance V of between 0.5 mm/s and 50 mm/s are possible in the embodiment shown. The linear movement device 30 is set up accordingly.

In the specifically shown coating of the catheter tube 100, a rotational speed U of between 300 rpm and 600 rpm, a speed of advance V of between 5 mm/s and 10 mm/s and an application rate of between 1 μl/s and 3 μl/s are provided, the viscosity V of the viscous coating solution S used in the present case being between 35×10⁻³ kg/ms and 55×10⁻³ kg/ms. The coating volume is between 1 μl/cm and 4 μl/cm.

In an embodiment not shown in the figures, the rotational speed U is 600 rpm, the speed of advance V is 10 mm/s and the application rate is between 0.9 μl/s and 2.5 μl/s, the viscosity V of the viscous coating solution S used in the present case being between 43×10⁻³ kg/ms und 48×10⁻³ kg/ms.

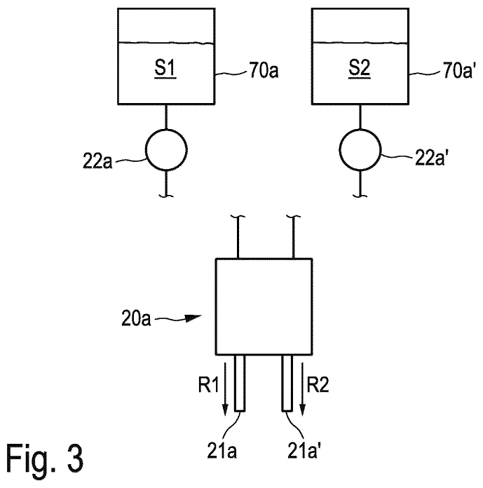

FIG. 3 shows a differently designed application device 20a, which can be used instead of the application device 20 in the apparatus 1 according to FIG. 1.

The application device 20a according to FIG. 3 is provided for applying a first solution component S1 and a second solution component S2, which together form the viscous coating solution or coating. Such multi-component coatings are generally known, especially in the hydrophilic coating of catheter tubes.

The first solution component S1 is stored in a first solution container 70a. The second solution component S2 is stored in a second solution container 70a′. The two solution containers 70a, 70a′ are connected to the application device 20a via a respective fluid line (without reference sign). In addition, separate pump units 22a, 22a′ are provided. Each fluid line, and thus each of the two solution containers 70a, 70a′, is assigned one of the two pump units 22a, 22a′. In the variant shown, the two pump units 22a, 22a′ are arranged away from the actual application device 20a and are stationary relative to this.

The application device 20a has a first application unit 21a and a second application unit 21a′. As regards the specific design of the two application units 21a, 21a′, what has already been mentioned as regards FIG. 1 and the application unit 21 shown therein applies mutatis mutandis. The first application unit 21a is provided for applying the first solution component S1 and is connected to the first solution container 70a via the fluid line not shown in any detail. The second application unit 21a′ is connected to the second solution container 70a′ via the further fluid line (without reference sign).

In the variant shown in FIG. 3, the first solution component S1 can be applied at a first application rate R1. The second solution component S2 can be applied at a second application rate R2. The two application rates R1, R2 can be changed/controlled via a corresponding control of the respective pump unit 22a, 22a′.

To form the coating, the two solution components S1, S2 are individually crosslinked. For this purpose, the apparatus 1 can have the optional crosslinking device 60 already explained with reference to FIG. 1 and provided as an alternative to the evaporation device 50. The crosslinking device 60 is configured for crosslinking and/or accelerating the crosslinking of the solution components S1, S2 applied to the lateral face. For this purpose, the crosslinking device 60 can, for example, have a light source, in particular a UV light source.

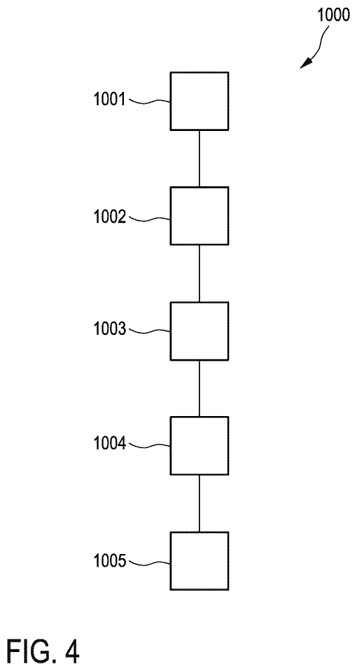

FIG. 4 schematically shows an embodiment of a method 1000 according to the invention. The method 1000 can be carried out using the apparatus 1 according to FIG. 1. The method 1000 entails clamping 1001 the catheter tube 100 in the rotation device 10. The method 1000 further entails rotating 1002 the clamped catheter tube 100 by means of the rotation device 10, the rotation taking place at the rotational speed U. The method 1000 further entails applying 1003 the viscous coating solution S to the lateral face 102 of the rotating catheter tube 100. The viscous coating solution S is applied here by means of the application device 20. This at the application rate R. The method 1000 further entails linearly moving 1004 the application device 20 along the longitudinal axis 101 of the rotating catheter tube 100. The application device 20 is moved linearly along the longitudinal axis 101 by means of the linear movement device 30 at a defined speed of advance V. The method 1000 further entails controlling 1005 the rotational speed U, the application rate R and the speed of advance V by means of the control device 40. In this case, the rotational speed U, the application rate R and the speed of advance V are controlled in a coordinated manner depending on the viscosity C of the viscous coating solution S, such that the viscous coating solution S is applied over the full surface of the lateral face 102 in the form of said helix H which overlaps along the longitudinal axis 101.

1.-15. (canceled)

16. An apparatus for coating a medical invasive component having a longitudinal axis and a lateral face which is rotationally symmetrical about the longitudinal axis and extends longitudinally in a straight manner, the apparatus comprising
a rotation device which is configured to clamp the invasive component and rotate the latter in a driven manner about its longitudinal axis at a defined rotational speed,
an application device which is rotationally immovable relative to the longitudinal axis and which is configured to apply a viscous coating solution onto the lateral face of the rotating invasive component at a defined application rate,
a linear movement device which is configured for driven relative linear movement between the application device and the rotation device at a defined speed of advance along the longitudinal axis of the rotating invasive component,
a control device which is configured to control the rotational speed of the rotation device, the application rate of the application device and the speed of advance of the linear movement device,
wherein the control device is configured to control the rotational speed, the application rate and the speed of advance in a coordinated manner depending on the viscosity of the viscous coating solution, such that the viscous coating solution can be applied over the full surface of the lateral face in the form of a helix which overlaps along the longitudinal axis.

17. The apparatus according to claim 16, wherein the viscous coating solution to be applied is formed from a plurality of separately stored solution components, wherein the application device is configured for simultaneous application of the plurality of solution components and has an application unit for each of the plurality of solution components.

18. The apparatus according to claim 16, wherein an evaporation device is present and is configured for evaporating and/or accelerating evaporation of a volatile solution component of the viscous coating solution applied to the lateral face.

19. The apparatus according to claim 16, wherein a crosslinking device is present and is configured for crosslinking and/or accelerating crosslinking of crosslinkable solution components of the viscous coating solution applied to the lateral face.

20. The apparatus according to claim 16, wherein the rotation device is configured for the clamping and driven rotation of invasive components having a diameter of 0.5 mm to 10 mm and a length of 40 mm to 500 mm and at a rotational speed of 20 rpm to 900 rpm, preferably at a rotational speed of 300 rpm to 600 rpm.

21. The apparatus according to claim 16, wherein the application device is configured for applying viscous coating solutions having a viscosity of 25×10⁻³ kg/ms to 80×10⁻³ kg/ms, preferably 35×10⁻³ kg/ms to 55×10⁻³ kg/ms, at an application rate of 0.5 μl/s to 10 μl/s, preferably 1 μl/s to 3 μl/s.

22. The apparatus according to claim 16, wherein the linear movement device is configured for the driven linear movement of the application device and/or of the rotation device at a speed of advance of between 0.5 mm/s and 50 mm/s, preferably of between 5 mm/s and 10 mm/s.

23. A method for coating a medical invasive component having a longitudinal axis and a lateral face which is rotationally symmetrical about the longitudinal axis and extends longitudinally in a straight manner, the method comprising the following steps:
clamping the invasive component into a rotation device;
rotating the clamped invasive component by means of the rotation device, the rotation taking place at a defined rotational speed;
applying a viscous coating solution to the lateral face of the rotating invasive component, wherein the viscous coating solution is applied by means of an application device, which is rotationally immovable relative to the longitudinal axis, and at a defined application rate;
linearly moving the application device and/or the rotation device along the longitudinal axis of the rotating invasive component, wherein the application device is moved linearly by means of a linear movement device and at a defined speed of advance relative to the rotation device, and/or vice versa;
controlling the rotational speed, the application rate and the speed of advance by means of a control device,
wherein the rotational speed, the application rate and the speed of advance are controlled in a coordinated manner depending on the viscosity of the viscous coating solution, such that the viscous coating solution is applied over the full surface of the lateral face in the form of a helix which overlaps along the longitudinal axis.

24. The method according to claim 23, wherein the rotational speed is between 20 rpm and 900 rpm, preferably between 300 rpm and 600 rpm.

25. The method according to claim 23, wherein the viscosity is between 25×10⁻³ kg/ms and 80×10⁻³ kg/ms, preferably between 35×10⁻³ kg/ms and 55×10⁻³ kg/ms.

26. The method according to claim 23, wherein the application rate is between 0.5 μl/s and 10 μl/s, preferably between 1 μl/s and 3 μl/s.

27. The method according to claim 23, wherein the speed of advance is between 0.5 mm/s and 50 mm/s, preferably between 5 mm/s and 10 mm/s.

28. The method according to claim 23, wherein the rotational speed is between 300 rpm and 600 rpm, wherein the viscosity is between 35×10⁻³ kg/ms and 55×10⁻³ kg/ms, wherein the application rate is between 1 μl/s and 3 μl/s, and wherein the speed of advance is between 5 mm/s and 10 mm/s.

29. The method according to claim 23, wherein, in relation to the gravitational direction, the viscous coating solution is discharged downward in the form of a hanging drop on the application device, wherein the hanging drop is contacted by the rotating invasive component, as a result of which the viscous coating solution winds helically around the lateral face.

30. The method according to claim 23, wherein, in relation to the gravitational direction, the viscous coating solution is discharged upward in the form of a standing drop on the application device, wherein the standing drop is contacted by the rotating invasive component, as a result of which the viscous coating solution winds helically around the lateral face.
